(19) United States
(12) Patent Application Publication
(10) Pub. No.: US 2008/0027421 A1
(43) Pub. Date: Jan. 31, 2008

(54) CryoBalloon Treatment for Postpartum Hemorrhage

(76) Inventor: David W. Vancelette, San Diego, CA (US)

Correspondence Address: AMS RESEARCH CORPORATION, 10700 BREN ROAD WEST, MINNETONKA, MN, 55343, US

(21) Appl. No.: 11/829,186

(22) Filed: Jul. 27, 2007

Related U.S. Application Data
(60) Provisional application No. 60/820,520, filed on Jul. 27, 2006.

(51) Int. Cl. A61B 18/02

(52) U.S. Cl. 606/21; 604/103.02; 606/193; 607/105

(57) ABSTRACT

Systems and related methods for treating postpartum hemorrhage through insertion of a flexible and expandable cryoballoon into the intrauterine cavity, inflating it to conform to the size and shape of the cavity, pressurizing it to apply pressure on damaged tissue and/or blood vessels that are bleeding, and filling it with a chilled fluid to provide numbing and blood coagulation. The cryoballoon can be coated with a drug or hormone to promote blood coagulation and/or uterine contractions to expedite the cessation of bleeding. In addition, cryoballoon can be coated with a topical anesthetic and/or antiseptic agent to numb and clean the damaged areas. Further, the cryoballoon can be fabricated of a biodegradable, bioerodeable or other biocompatible material so that it can be left in the intrauterine cavity for an extended period of time after insertion.

### PRIORITY CLAIM

The present application claims priority to U.S. Provisional Application No. 60/820,520, filed Jul. 27, 2006 and entitled “CRYOBALLOON TREATMENT FOR POSTPARTUM HEMORRHAGE”, which is herein incorporated by reference in its entirety.

### FIELD OF THE INVENTION

The present disclosure relates to treatment for postpartum hemorrhage and more particularly to a cryoballoon system and method used to treat postpartum hemorrhage.

### BACKGROUND OF THE INVENTION

Postpartum hemorrhage (“PPH”) is a potentially life threatening complication of both vaginal and cesarean delivery. PPH is most commonly caused by uterine atony in which the uterus fails to contract normally after the delivery of the baby. Any bleeding that results in signs and symptoms of hemodynamic instability, or that could result in hemodynamic instability if untreated, is considered PPH. Such excess and rapid blood loss can cause a severe drop in the mother's blood pressure and may lead to shock and death if not treated. PPH is one of the leading causes of maternal deaths in the United States and world wide. Techniques for managing PPH can be medical, mechanical, or surgical.

### SUMMARY OF THE INVENTION

The present disclosure is directed to systems and related methods for treating postpartum hemorrhage. Postpartum hemorrhage can be treated by inserting a flexible and expandable cryoballoon into the intrauterine cavity, inflating it to conform to the size and shape of the cavity, pressurizing it to apply pressure on damaged tissue and/or blood vessels that are bleeding, and filling it with a chilled fluid to provide numbing and blood coagulation. In some representative embodiments, the cryoballoon can be coated with a drug or hormone to promote blood coagulation and/or uterine contractions to expedite the cessation of bleeding. In addition, cryoballoon can be coated with a topical anesthetic and/or antiseptic agent to numb and clean the damaged areas. Further, the cryoballoon can be comprised of a biodegradable, bioerodeable or other biocompatible material so that it can be left in the intrauterine cavity for an extended period of time after insertion.

In one aspect of the present disclosure, a cryoballoon system provides a cryoballoon that is inflated and filled with a chilled fluid by an inflation bulb. Cryoballoon can be inserted into the intrauterine cavity through a retractable delivery sheath. A chilled fluid can then be introduced into the inflation bulb and pumped through a lumen to fill the cryoballoon, applying pressure to and freezing the intrauterine cavity.

In another aspect of the present disclosure, a cryoballoon system provides a cryoballoon that can be filled, pressurized, and chilled with a compressed fluid provided in a can or other container for a single use application. The fluid can be released from the container and travel through a one-way valve and a channel into the cryoballoon. The cryoballoon and channel can then be detached from the container. The one-way valve can remain in the channel to ensure the balloon remains inflated and pressurized.

In another aspect of the present disclosure, methods for treating postpartum hemorrhage with a cryoballoon system are disclosed. Generally speaking, an uninflated cryoballoon can be positioned within the intrauterine cavity such that a pressurized and/or chilled fluid can be utilized to inflate the cryoballoon such that damaged tissue can be exposed to pressure and/or cool temperatures. The use of pressure and/or cool temperatures can assist in numbing tissue as well as promoting blood coagulation.

In yet another aspect of the present disclosure, a cryoballoon system provides a cryoballoon attached to a tip portion of a cryoprobe used in a cryosurgical system. Cryosurgical system circulates a chilled and/or pressurized fluid through a cryoprobe. Tip portion of cryoprobe can have apertures through which the fluid is released to fill the cryoballoon.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a view of an embodiment of a cryoballoon system according to the present disclosure.

FIG. 2 is a view of an embodiment of a cryoballoon system according to the present disclosure.

FIG. 3 is a view of an embodiment of a cryoballoon system according to the present disclosure.

FIG. 4 is a view of an embodiment of a cryoballoon system according to the present disclosure.

FIG. 5 is a view of an embodiment of a cryoballoon system according to the present disclosure.

FIG. 6 is a view of an embodiment of a cryoballoon system according to the present disclosure.

FIG. 7 is a view of a cryosurgical system with which an embodiment of a cryoballoon system according to the present disclosure can be used.

### DETAILED DESCRIPTION OF THE PREFERRED EMBODIMENTS

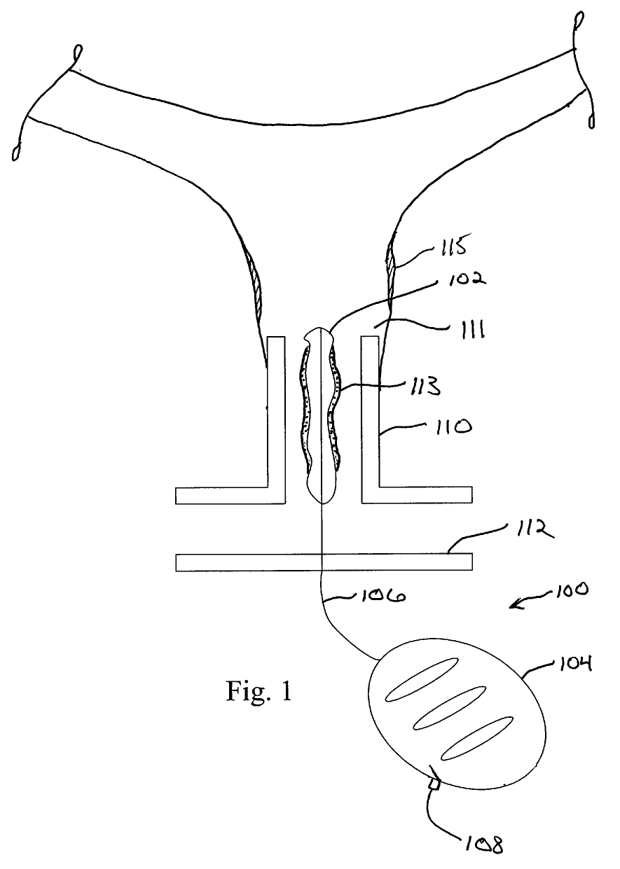

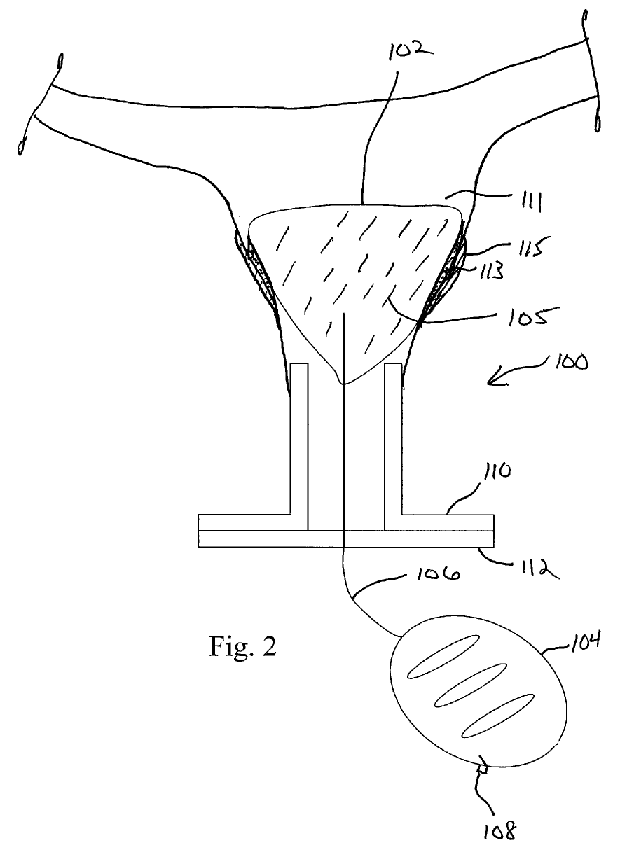

Referring to FIGS. 1 and 2, there is illustrated an embodiment of a cryoballoon system 100 according to the present disclosure. Cryoballoon system 100 includes a flexible and expandable cryoballoon 102 that can be inflated with an inflation bulb 104. A chilled and/or pressurized cryogenic fluid 105, for example saline, can be introduced into inflation bulb 104, and then pumped through a lumen 106 into cryoballoon 102. A check valve 108 such as, for example, a flapper-style check valve or other suitable one way valve, can be used to permit the introduction of fluid into the inflation bulb 104 while at the same time preventing the fluid from escaping the inflation bulb 104. In this manner, a medical professional can maintain the pressure within the cryoballoon 102.

Before inflation, a retractable delivery sheath 110 can be inserted into the intrauterine cavity 111 to provide a path for insertion of the cryoballoon 102. Cryoballoon system 100 can be provided with a handle 112 that rests against retractable delivery sheath 110 upon insertion to ensure that cryoballoon 102 is neither over inserted nor under inserted with respect to the intrauterine cavity. Following insertion, cryoballoon 102 can be inflated with the chilled and/or pressurized fluid using the inflation bulb 104 as described above to conform to the intrauterine cavity. The pressurized and/or chilled fluid within cryoballoon 102 provides numbing and promotes blood coagulation of damaged tissue 115 and/or blood vessels that are bleeding as a result of PPH. Inflation bulb 104 can also be provided with a standard release valve to deflate the cryoballoon 102 and allow its removal subsequent to treatment. In some embodiments, cryoballoon 102 can comprise a biodegradable, bioerodeable or other long term bio-compatible material such that the cryoballoon 102 can remain within the intrauterine cavity 111 for an extended period of time upon insertion.

In some representative embodiments, an exterior portion of cryoballoon 102 can be coated with various treatment agents 113 to improve the ease and effectiveness of the treatment. In one representative embodiment, treatment agent 113 can comprise a coagulation agent. Alternatively, treatment agent 113 can comprise a contraction stimulating agent such as, for example, oxytocic drugs including oxytocin, ergonovine, methylergonovine, carboprost and misoprostoal. In yet another embodiment, treatment agent 113 can comprise a numbing agent. Treatment agent 113 can further comprise a cleansing agent such as, for example, an antiseptic. Finally, treatment agent 113 can further comprise a suitable antibiotic.

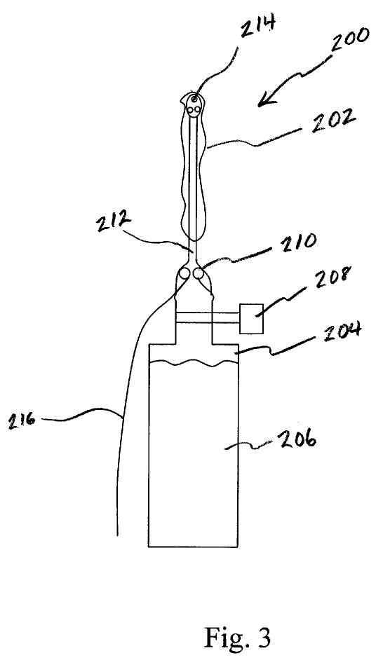

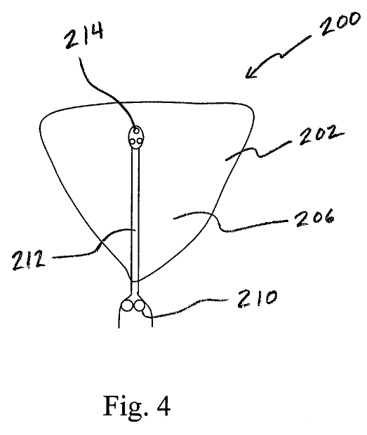

Referring now to FIGS. 3 and 4, there is illustrated another embodiment of a cryoballoon system 200 according to the present disclosure. Cryoballoon system 200 can include a flexible and expandable cryoballoon 202 that can be filled with a chilled and/or pressurized fluid 206 contained in a canister 204 or other suitable container.

Cryoballoon 202 can be situated over a distal end 207 of a fluid channel 212. A proximal end 209 of fluid channel 212 is operably connected to canister 204 such that a fluid pathway 211 is defined between the cryoballoon 202 and the canister 204. Upon insertion of the cryoballoon 202 into the intrauterine cavity, a valve assembly 208 can be turned to release the fluid 206 through a check valve 210, or other one way valve, and into the channel 212. Fluid 206 flows through the fluid pathway 211 and is released into the cryoballoon 202 through a plurality of dispensing apertures or vent holes 214 located at distal end 207 of fluid channel 212. Cryoballoon 202 can optionally be inserted with the aid of the retractable delivery sheath 110, as described above. A vent line 216 can be connected to cryoballoon system 200 to ensure that the cryoballoon 202 is not over inflated. In some representative embodiments, cryoballoon system 200 can be provided with an auto-pressure sensing means to ensure the cryoballoon 202 is not over inflated, and in some situations to automatically vent cryoballoon 202 through vent line 216.

Once the cryoballoon 202 is inflated, the connection with canister 204 can be broken at fluid channel 212 and the canister 204 can be discarded. The check valve 210 can remain within the fluid channel 212 to ensure that the cryoballoon 202 remains inflated and that fluid is not released through fluid channel 212. Pressure can be released from the cryoballoon 202 so that it can be deflated and removed by squeezing channel 212 to allow fluid 206 to flow past the check valve 210.

Cryoballoon 202 can additionally be coated on an exterior portion of the cryoballoon 202 with treatment agent 113 to further assist in treatment of damaged tissue 115. Similarly to cryoballoon 102, cryoballoon 202 can comprise a biodegradable, bioerodeable or bio-compatible material such that the cryoballoon 202 can remain in the intrauterine cavity 111 for an extended period of time subsequent to insertion.

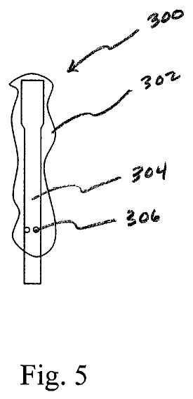

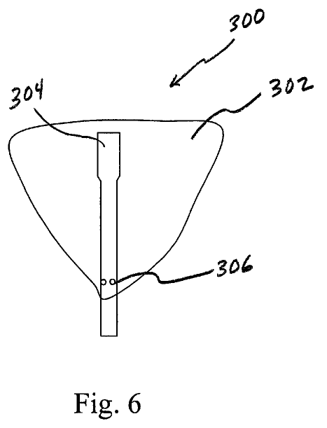

Referring to FIGS. 5 and 6, another embodiment of a cryoballoon system 300 according to the present disclosure is illustrated. Cryoballoon system 300 can include a flexible and expandable cryoballoon 302 on a cryoprobe tip portion 304 of a cryosurgical system. Cryoprobe tip portion 304 can include one or more apertures 306 through which fluid can enter cryoballoon 302.

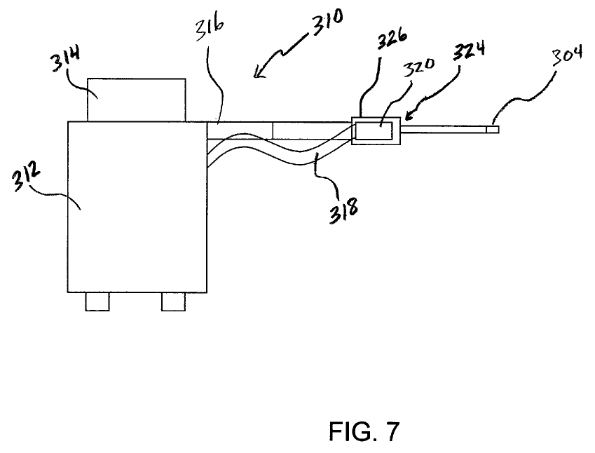

A representative cryosurgical system that can be used with the embodiment of the present invention depicted in FIGS. 5 and 6 is illustrated in FIG. 7. Cryosurgical system 310 can include a refrigeration and control console 312 with an attached display 314. A chilled and/or pressurized fluid, such as, for example, saline or a mixed gas refrigerant, can be transferred from control console 312 to a cryostat heat exchanger module 320 through a flexible line 318. The cryostat heat exchanger module 320 can be located within a handle 326 of a cryoprobe 324 having a tip portion 304. The cryoprobe 324 can also be connected to the control console 312 by way of an articulating arm 316, which may be manually or automatically used to position the cryoprobe 324. Although depicted as having the flexible line 318 as a separate component from the articulating arm 316, cryosurgical system 310 may incorporate the flexible line 318 within the articulating arm 316.

To utilize cryoballoon system 300 with cryosurgical system 310, a cryoballoon 300 can be attached and sealed to a cryoprobe tip portion 304 of an existing cryoprobe 314. Alternatively, cryoprobe 314 may be specially formed with an integral cryoballoon 300. When the chilled fluid flows into cryoprobe tip portion 304, it flows through apertures 306 and fills cryoballoon 302, providing numbing and imparting pressure onto the intrauterine cavity. Cryoballoon system 300 can also be provided with a tube to allow the fluid to be drained out of the intrauterine cavity after treatment. Alternatively, the fluid can be recirculated back to the control console 312 to be re-cooled and/or re-pressurized for a subsequent procedure.

Cryoballoon 302 can be coated with treatment agent 113 to promote healing and treatment of damaged tissue 115. Cryoballoon 302 can comprise a biodegradable, bioerodeable or bio-compatible material such that the cryoballoon 302 can remain in the intrauterine cavity 111 for an extended period of time subsequent to insertion.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A system for treating postpartum hemorrhage, comprising:
an expandable balloon having an exterior portion coated with a treatment agent,
an inflation bulb;
a lumen fluidly connecting the expandable balloon with the inflation bulb; and
a cryogenic fluid introduced into the expandable balloon with the inflation bulb such that the exterior portion of the expandable balloon is in contact with a patient's uterine cavity to deliver the treatment agent and wherein the cryogenic fluid is chilled to a sufficient level to numb and promote blood coagulation of damaged tissue within the uterine cavity.

2. The system of claim 1, wherein the treatment agent comprises a coagulant.

3. The system of claim 1, wherein the treatment agent comprises a numbing agent.

4. The system of claim 1, wherein the treatment agent comprises an antibiotic.

5. The system of claim 1, wherein the treatment agent comprises an antiseptic.

6. The system of claim 1, wherein the treatment agent comprises a contraction stimulating agent.

7. The system of claim 1, wherein the inflation bulb includes a one-way valve configured to allow cryogenic fluid to enter the inflation bulb but prevent fluid from escaping the inflation bulb.

8. The system of claim 7, wherein the inflation bulb includes a release valve configured to allow deflation of the balloon.

9. The system of claim 1, further comprising a retractable delivery sheath positioned over the expandable balloon and lumen, the retractable delivery sheath providing a pathway for the expandable balloon and lumen into the uterine cavity.

10. The system of claim 1, wherein the expandable balloon comprises a biodegradable, bioerodeable or bio-compatible polymer.

11. A system for treating postpartum hemorrhage, comprising:
a refrigeration and control console;
a cryogenic fluid;
a cryoprobe having a tip portion, said tip portion having a plurality of apertures; and
a flexible balloon sealed over the tip portion of cryoprobe, the flexible balloon having an exterior portion coated with a treatment agent;
wherein the cryogenic fluid is chilled within the refrigeration and control console, and wherein the cryogenic fluid is dispensed into the flexible balloon through the plurality of apertures such that the flexible balloon expands within a patient's uterine cavity to deliver the

12. The system of claim 11, wherein upon completion of treatment, the cryogenic fluid is withdrawn from the flexible balloon and returned to the console for reuse in a subsequent treatment.

13. The system of claim 11, wherein the treatment agent is selected from the group consisting of: a coagulant; a numbing agent, an antibiotic, an antiseptic and a contraction stimulating agent.

14. The system of claim 11, wherein the flexible balloon comprises a biodegradable, bioerodeable or bio-compatible polymer.

15. A method of treating postpartum hemorrhage, comprising:
inserting an expandable balloon into a patient's intrauterine cavity;
inflating the expandable balloon with a cryogenic fluid until the expandable balloon conforms to the cavity;
administering a treatment agent on an external surface of the expandable balloon to damaged tissue through contact of the external surface with the damaged tissue; and
chilling the damaged tissue to numb the intrauterine cavity and promote blood coagulation of damaged tissue; in the intrauterine cavity.

16. The method of claim 15, further comprising:
positioning a retractable delivery sheath within the intrauterine cavity; and
advancing the expandable balloon through the delivery sheath in into the intrauterine cavity.

17. The method of claim 15, further comprising:
fabricating the expandable balloon from a biodegradable, bioerodable or biocompatible material.

18. The method of claim 15, further comprising:
deflating the expandable balloon by removing the cryogenic fluid.

19. The method of claim 18, further comprising:
leaving the deflated expandable balloon in the intrauterine cavity.

20. The method of claim 18, further comprising:
reusing the cryogenic fluid in a subsequent treatment.
